(19) United States
(12) Patent Application Publication
(10) Pub. No.: US 2005/0048042 A1
(43) Pub. Date: Mar. 3, 2005

(54) Compositions and methods for the treatment of schizophrenia and addictive disorders

(75) Inventors: Boris Tabakoff, Denver, CO (US); Michael Zawada, Denver, CO (US); Masami Yoshimura, Denver, CO (US); Paula Hoffman, Denver, CO (US)

Correspondence Address: MEDLEN & CARROLL, LLP, Suite 350, 101 Howard Street, San Francisco, CA, 94105, US

(73) Assignee: The Regents of the University of Colorado, Boulder, CO (US)

(21) Appl. No.: 10/877,654

(22) Filed: Jun. 24, 2004

Related U.S. Application Data
(60) Provisional application No. 60/482,338, filed on Jun. 24, 2003.

(51) Int. Cl. A61K 48/00; C12N 5/08

(52) U.S. Cl. 424/93.21; 435/368

(57) ABSTRACT

The present invention relates to compositions and methods for regulating neurotransmitter activity in the brains of patients with schizophrenia and addictive disorders. In particular, the present invention provides treatments directed at reducing dopaminergic overactivity.

This application claims the benefit of U.S. Provisional Application No. 60/482,338, filed on Jun. 24, 2003.

This invention was made in part with government support under grant AA13473, from the National Institute on Alcohol Abuse and Alcoholism. As such, the United States Government has certain rights in the invention.

### FIELD OF THE INVENTION

The present invention relates to compositions and methods for regulating neurotransmitter activity in the brains of patients with schizophrenia and addictive disorders. In particular, the present invention provides treatments directed at reducing dopaminergic overactivity.

### BACKGROUND OF THE INVENTION

Schizophrenia is a major, chronic brain disorder characterized by psychotic symptoms and deficits in normal cognitive, emotional and social functioning (See, e.g., Hyman, “Schizophrenia,” in Scientific American Medicine, volume 3, 2000). Psychotic symptoms associated with schizophrenia include delusions, hallucinations, disorganized speech, and grossly disorganized or catatonic behavior. These symptoms are frequently categorized as positive or negative symptoms (Diagnostic and Statistical Manual of Mental Disorders, 4th edition, American Psychiatric Association, Washington DC, 1994; DSM-IV). Positive symptoms such as delusions and hallucinations, are thoughts, perceptions, emotions and behaviors that are not present in normal individuals. In contrast, negative symptoms include the absence of normal human responses and behaviors.

Schizophrenia is one of the most common psychotic disorders, affecting 1% of the world population. The cost to society is large in that patients with schizophrenia constitute both a significant proportion of the totally and permanently disabled, and a large fraction of the homeless population (Hyman, supra). This cost is very great, due in part to the relatively early onset of schizophrenia, which is typically between 15 to 30 years of age.

Antipsychotic drugs used to treat schizophrenia are typically chosen based upon the side effects a given patient is most likely to best tolerate. Undesirable side effects of antipsychotic drugs include anti-cholinergic side effects (e.g., dry mouth, constipation, blurred vision and urinary retention), extrapyramidal side effects, sedation and hypotension. Extrapyramidal side effects are movement disorders such as dystonia, parkinsoniism, akathisia, and tardive dyskinesia). Although the newer atypical antipsycliotic drugs cause extrapyramidal side effects less frequently than older medications, their ability to combat negative symptoms of schizophrenia is questionable. In addition, some of the atypical antipsychotic medications can cause blood dyscrasias. Thus, there remains a need in the art for improved treatments of schizophrenia which are unaccompanied by debilitating side effects leading to poor patient compliance. In particular, treatments directed to remedying neurotransmitter dysregulation in patients with schizophrenia or addictive disorders are contemplated to reduce morbidity and mortality associated with these conditions.

### SUMMARY OF THE INVENTION

The present invention relates to compositions and methods for regulating neurotransmitter activity in the brains of patients with schizophrenia and addictive disorders. In particular, the present invention provides treatments directed at reducing dopaminergic overactivity.

In the first place, the present invention provides compositions comprising a neural stem cell engineered to express a mammalian dopamine transporter. In some embodiments, the neural stem cell constitutively expresses the dopamine transporter, while in other embodiments, the neural stem cell inducibly expresses the dopamine transporter. In a subset of these embodiments, the dopamine transporter is a human transporter comprising the amino acid sequence set forth as SEQ ID NO:1. Also provided are compositions that further comprise a physiologically acceptable diluent.

In addition, the present invention provides methods of producing a neural stem cell which inducibly expresses a dopamine transporter, comprising: providing: i) at least one neural stem cell, ii) an expression vector comprising a dopamine transporter nucleic acid sequence in operable combination with a tetracycline-responsive transcription activator and a promoter, and iii) at least one inducible vector comprising a tetracycline-responsive transcription activator and a tetracycline-controlled transcriptional silencer; and contacting the at least one neural stem cell with the expression vector and the at least one inducible vector under conditions suitable for producing a dopamine transporter-positive neural stem cell which inducibly expresses the dopamine transporter. In some embodiments, the dopamine transporter is a human dopamine transporter comprising the amino acid sequence set forth as SEQ ID NO:1. In preferred embodiments, at least one of the vectors further comprises a drug selection marker. In particularly preferred embodiments, the dopamine transporter-positive neural stem cell internalizes dopamine after growth in the presence of an inducer selected from the group consisting of tetracycline and a tetracycline derivative. In further preferred embodiments, the dopamine transporter-positive neural stem cell is suitable graft material for human transplantation.

Moreover, the present invention provides methods of alleviating at least one symptom of schizophrenia in a schizophrenic patient, comprising: providing a neural stem cell engineered to inducibly express a human dopamine transporter; and transplanting the neural stem cell into the forebrain of a schizophrenic patient, under conditions suitable for alleviating at least one symptom of schizophrenia. In some embodiments, the at least one symptom comprises a symptom selected from the group consisting of a positive symptom, a negative symptom, a cognitive symptom, and a mood symptom. In various embodiments, the positive symptom is selected from the group consisting of delusions, hallucinations, disorganized speech, and catatonia, the negative symptom is selected from the group consisting of affective flattening, alogia, avolition and anhedonia, the cognitive symptom is selected from the group consisting of an attention deficit, poor memory recall, and a reduced executive function, while the mood symptom is selected from the group consisting of dysphoria, suicidality, and hopelessness. In particularly preferred embodiments, the patient has drug treatment-refractory schizophrenia. Also provided are methods in which the alleviating is assessed by DSM-IV diagnostic criteria or ICD-10 diagnostic criteria.

Additionally, the present invention provides methods of reducing the reinforcing effects of an addictive drug in a patient with an addictive disease comprising: providing a neural stem cell engineered to inducibly express a human dopamine transporter; and transplanting the neural stem cell into the brain of a patient with an addictive disease, under conditions suitable for reducing the reinforcing effects of an addictive drug. In some embodiments, the addictive drug is selected from the group consisting of cocaine, an opiate, nicotine, a cannabinoid, a barbiturate, a benzodiazepine, and alcohol. In preferred embodiments, the addictive disease is life-disrupting or life-threatening. In particularly preferred embodiments, the reducing comprises lessening the amount of the addictive drug intake by the patient and/or lessening the frequency of the addictive drug intake by the patient.

Although some preferred embodiments comprise enhancing the function of the dopamine transporter by delivery of genetically engineered stem cells, the present invention is not limited to transplantation of transfected stem cells. Some alternative embodiments comprise enhancing the expression and/or function of the dopamine transporter in the brain by for instance, infection with viral vectors engineered to express the dopamine transporter. Other means of accomplishing delivery and expression of the dopamine transporter gene in the brain are also suitable for use with the present invention.

### DESCRIPTION OF THE FIGURES

FIG. 1 provides flow charts of the generation of C17.2 HDAT cells in panel A, and of the generation of Tet-On C17.2 hDAT cells in panel B.

FIG. 2 provides a map of the pCAIP2-hDAT plasmid from which expression of FLAG-HA-hDAT is driven.

FIG. 3 provides a map of the pTet-On-PUR plasmid in panel A, and a map of the pTet-tTS plasmid in panel B. The plasmid pTet-On-PUR expresses the reverse tetracycline responsive transcription activator (rtRA), while the plasmid pTet-tTS expresses the tetracycline-controlled transcriptional silencer (tTS).

FIG. 4 provides a map of the pBI-EGFP-hDAT plasmid from which expression of both FLAG-HA-HDAT and EGFP is driven. This plasmid contains a bidirectional promoter consisting of the tetracycline-responsive element (TRE) and two identical minimal cytomegalovirus promoters (PminCMV). PminCMV-1 controls the expression of HDAT, while pminCMV-2 controls the expression of EGFP.

FIG. 5 depicts expression of enhanced green fluorescent protein (EGFP) by cells transfected with pBI-EGFP-hDAT, in the absence (no Dox) and in the presence (Dox) of 1 μg/ml doxycycline.

FIG. 6 shows the amino acid sequence (SEQ ID NO:1) of the human dopamine transporter (hDAT), which is encoded by the nucleic acid sequence (SEQ ID NO:2) disclosed as GENBANK Accession No. NM_{—}001044.

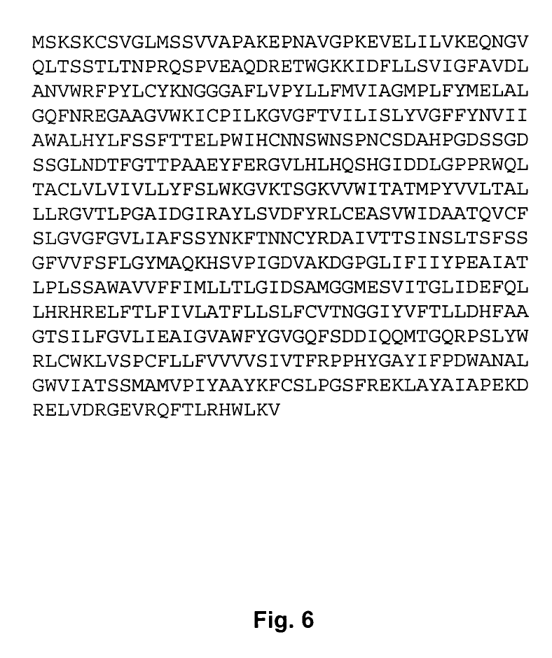

FIG. 7 graphically depicts a comparison of dopamine uptake by hDAT-transfected cells in the absence (no Dox) and in the presence (Dox) of 1 μg/ml doxycycline.

FIG. 8 shows transplanted cells one week after grafting, identified by X-gal staining, in a coronal section of a transplanted brain at the level of the nucleus accumbens. Panel A shows that bilateral grafts of naive C17.2 cells reside in the nucleus accumbens. Panels B and C provide magnified views of the area inside the box in the previous panel.

FIG. 9 shows that one week after grafting, C17.2 HDAT cells express hDAT in the grafts, while control (naive) C17.2 cells do not. Panels A-D show coronal brain sections with control C17.2 cells in a transplant tract in the striatum. Panels A and B are low and high magnification images showing beta-galactosidase positive cells, while panels C and D are low and high magnification images, showing that the transplanted hDAT-negative cells stand out against a background of endogenous mDAT visualized by immunohistochemistry. Panels E-H show coronal brains sections of bilateral transplants of hDAT-expressing C17.2 cells in the nucleus accumbens. Panels E and F are low and high magnification images of beta-galactosidase positive cells, while panels G and H are low and high magnification images of hDAT-expressing cells in the transplant tract. Scale bar is: 2 mm for panels A, C, E and G; 200 μm for panels B and F; and 400 μm for panels D and H.

FIG. 10 shows that when alcohol preference (intake of alcohol solution versus water) was determined over a 3-day period (days 3-6) after surgery (“post”), there was a significantly (*p<0.05, paired t-test) lower level of alcohol preference in animals that had received the C17.2 hDAT cells (bar labeled b7), in comparison to sham-operated animals (bar labeled s), or animals that received hDAT-negative C17.2 cells (bar labeled f2).

### GENERAL DESCRIPTION OF THE INVENTION

The primary proposed brain malfunction in schizophrenia is an overactivity of dopaminergic systems in the forebrain limbic areas. Thus, treatments directed at reducing dopamine levels in the brains of schizophrenic patients are contemplated to be useful in alleviating symptoms associated with this disease. As described herein, mammalian cells have been engineered to overexpress the dopamine transporter, making these cells capable of taking up and inactivating relatively large amounts of dopamine. Similarly, human embryonic stem cells are engineered to overexpress the dopamine transporter. Implantation of these engineered cells into the forebrains of schizophrenic individuals is contemplated to result in clinical improvement in the signs and symptoms of schizophrenia.

With regard to addictive disorders, most addictive drugs, such as cocaine, opiates, nicotine, cannabinoids, and central nervous system depressants (including barbiturates, benzodiazepines and alcohol), activate the mesolimbic dopaminergic neurons of the forebrain. This activation generates as increase in dopamine release in areas such as the nucleus accumbens and other regions of the extended amygdala. This dopamine release is related to the reinforcing properties of addictive drugs, which in turn perpetuate drug-taking behavior. Thus, implantation of stem cells engineered to overexpress the dopamine transporter is contemplated to be valuable in the treatment of addictive disorders. Specifically, implantation of stem cells in areas of the brain important for the reinforcing effects of addictive drugs is contemplated. This type of therapeutic approach is contemplated to dampen or eliminate the overactivity of dopamine systems related to drug-taking behavior and thus would result in extinction of this behavior in drug-addicted individuals (elimination of drug-induced reinforcement will lead to extinction of the drug-taking behavior).

Definitions

To facilitate understanding of the invention, a number of terms are defined below.

The terms “subject” as used herein, refers to a human. It is intended that the term encompasses healthy individuals, as well as, individuals predisposed to, or suspected of having a psychiatric disorder. Typically, the terms “subject” and “patient” are used interchangeably. In some preferred embodiments of the present invention, the term subject refers to specific subgroups of patients including but not limited to schizophrenic individuals, alcohol/drug addicted individuals and/or persons suffering from comorbid addictive and other psychiatric disorders.

The term “gene” refers to a nucleic acid (e.g., DNA) sequence that comprises coding sequences necessary for the production of a polypeptide (e.g., DAT), precursor, or RNA (e.g., mRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segents of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. In particular, the terms “dopamine transporter gene,” “DAT gene,” “dopamine transporter nucleic acid,” and “DAT nucleic acid,” refer to the full-length DAT nucleotide sequence. The terms “DAT gene” and “DAT nucleic acid” as used herein, also encompass fragments of the DAT sequence, as well as other domains within the full-length DAT nucleotide sequence. Furthermore the term “DAT nucleotide sequence” encompasses DNA, cDNA, and RNA (e.g., GENBANK Accession No. NM_{—}001044; and SEQ ID NO:2) sequences.

In addition to containing introns, genomic forms of a gene may also include sequences located on both the 5′ and 3′ end of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

The term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “Southern blot,” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

The term “Northern blot,” as used herein refers to the analysis of RNA by electrophoresis of RNA on agarose gels to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein, the term “Tₘ” is used in reference to the “melting temperature.” The melting temperature is the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization, 1985). Other references include more sophisticated computations that take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under ‘medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

“High stringency conditions” when used in reference to nucleic acid hybridization comprise conditions equivalent to binding or hybridization at 42° C in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42° C. when a probe of about 500 nucleotides in length is employed.

“Medium stringency conditions” when used in reference to nucleic acid hybridization comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42° C. when a probe of about 500 nucleotides in length is employed.

“Low stringency conditions” comprise conditions equivalent to binding or hybridization at 42° C. in a solution consisting of 5×SSPE (43.8 g/l NaCl, 6.9 g/l NaH₂PO₄ H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 μg/ml denatured salmon sperm DNA followed by washing in a solution comprising 5×SSPE, 0.1% SDS at 42° C. when a probe of about 500 nucleotides in length is employed.

The art knows well that numerous equivalent conditions may be employed to comprise low stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of low stringency hybridization different from, but equivalent to, the above listed conditions. In addition, the art knows conditions that promote hybridization under conditions of high stringency (e.g., increasing the temperature of the hybridization and/or wash steps, the use of formamide in the hybridization solution, etc.) (see definition above for “stringency”).

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

As used herein, the term “sample template” refers to nucleic acid originating from a sample that is analyzed for the presence of “target.” In contrast, “background template” is used in reference to nucleic acid other than sample template that may or may not be present in a sample. Background template is most often inadvertent. It may be the result of carryover, or it may be due to the presence of nucleic acid contaminants sought to be purified away from the sample. For example, nucleic acids from organisms other than those to be detected may be present as background in a test sample.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

The term “sense primer” refers to an oligonucleotide capable of hybridizing to the noncoding strand of gene. The term “antisense primer” refers to an oligonucleotide capable of hybridizing to the coding strand of a gene.

As used herein, the term “fluorescent tag” refers to a molecule having the ability to emit light of a certain wavelength when activated by light of another wavelength. “Fluorescent tags” suitable for use with the present invention include but are not limited to green fluorescent protein, red fluorescent protein, and enhanced green fluorescent protein.

The term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “polymerase chain reaction” (“PCR”) refers to the method of K. B. Mullis. U.S. Pat. Nos. 4,683,195 4,683,202, and 4,965,188, hereby incorporated by reference, describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” as used herein, refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the term “sample” is meant to include a specimen obtained from subject. The term “sample” encompasses fluids, solids, and tissues. In preferred embodiments, the term “sample” refers to blood or biopsy material obtained from a living body for the purpose of examination via any appropriate technique (e.g., needle, sponge, scalpel, swab, etc.).

The term “schizophrenia” as used herein refers to a major mental disorder featuring psychotic symptoms during some phase of the illness, a long term course and a deterioration in function. Schizophrenic symptoms can be classified as positive, negative, cognitive and mood symptoms, which together or separately may result in behavioral disturbances (e.g., bizarre, apparently purposeless and stereotyped activity or inactivity). Various embodiments of the present invention are contemplated to effectively treat all four subtypes of schizophrenia, including catatonic, disorganized, paranoid and undifferentiated. In addition, the compositions and methods of the present invention are also contemplated to benefit patients with schizoid personality disorder (socially distant, detached) and patients with schizotypal personality disorder (odd, eccentric).

As used herein, the term “positive symptoms” refers to symptoms including but not limited to hallucinations (e.g., hearing voices), delusions (e.g., of persecution or grandiosity), disorganized speech and thought, altered sense of self and bizarre behavior. They are called positive symptoms because they are added on to the individuals experience and behavior.

The term “negative symptoms” as used herein, refers to deficit symptoms, including experience and behavior that should be there and is not. Negative symptoms include but are not limited to loss of motivation, flattened emotions, withdrawal from an active social life, poverty of thought and speech, and loss of former interests and pleasures.

As used herein, the term “cognitive symptoms,” refers to symptoms associated with a loss of cognitive ability including but not limited to attention deficits, memory loss, inability to plan for the future and poor capacity for abstract thought.

The term “mood symptoms” as used herein, refers to symptoms associated with a disturbed state of mind or predominant emotion such as dysphoria.

As used herein the terms “addictive disorder” and “substance-related disorder” refers to a disease characterized by the habitual psychological and physiologic dependence on a substance or practice that is beyond voluntary control. The term “addictive disorder” includes but is not limited to alcohol dependence (alcoholism), amphetamine dependence (stimulants, speed, uppers, diet pills), cannabis dependence (marijuana, grass, pot, weed, reefer, hashish, bhang, ganja), cocaine dependence (coke, crack, coca leaves), hallucinogen dependence (psychedelics, LSD, mescaline, peyote, psilocybin, DMT), inhalant dependence (sniffing: glue, gasoline, toluene, solvents), nicotine dependence (tobacco), opioid dependence (heroin, methadone, morphine, demerol, percodan, opium, codeine, darvon), phencyclidine dependence (PCP, angel dust), and sedative dependence (sleeping pills, barbiturates, seconal, valium, librium, ativan, xanax, quaaludes).

Specifically, the term “alcohol dependence” as used herein refers to a clinical syndrome (See, DSM-IV) that includes at least three of the following over 1 year: tolerance (e.g., increased drinking to achieve same effect); alcohol withdrawal signs; drinking more than intended; unsuccessful attempts to cut down on use; excessive time related to alcohol (e.g., obtaining, hangover); impaired social or work activities due to alcohol; and use despite physical or psychological consequences.

The terms “neural stem cells” “neural progenitors” and “neural precursor cells” as used herein refer to a relatively undifferentiated population(s) of cells in the central nervous system (i.e., brain and spinal cord), which have the ability to give rise to a broad array of specialized cells including both neurons and glial cells. Neural stem cells suitable for use with the methods and compositions of the present invention are obtained from harvested from post-natal, post-mortem, human brain and spinal cord tissue as made available for instance from the National Human Neural Stem Cell Resource (Children's Hospital of Orange County, Calif.).

As used herein, the terms “transplant cells” and “graft material” refer broadly to the component (e.g., tissue or cells) being grafted, implanted or transplanted. As used herein, the term “transplantation” refers to the transfer or grafting of tissues or cells from one part of a subject to another part of the same subject, or to another subject, or the introduction of biocompatible materials into or onto the body. As used herein, a transplanted tissue may comprise a collection of cells of identical or similar composition, or derived from an organism (i.e., a donor), or from an in vitro culture (i.e., a tissue culture system). The term “suitable graft material” refers to tissue with the desired phenotype (e.g., capable of constitutive or inducible expression of the dopamine transporter), which is free of deleterious contaminants (e.g., free of bacteria and fungi).

The term “recipient of transplanted cells” as used herein, refers broadly to the subject undergoing transplantation and receiving transplanted cells.

As used herein, the terms “neuron” and “nerve cell” refer to an excitable cell specialized for the transmission of signals within the central nervous system. A typical neuron consists of a cell body, an axon, axon terminals, and dendrites. Signals are transmitted in the form of neurotransmitters from the axon of one nerve cell to the dendrite of another nerve cell across a junction known as a synapse,

As used herein, the term “purified” refers to molecules, either nucleic or amino acid sequences or cells that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. Similarly, an “isolated hDAT-neural stem cell” is a cell purified from cell culture components including but not limited to hDAT-negative neural stem cells, fetal calf serum, etc.

The term “constitutively expresses” refers to the continuous expression of a gene of interest without any regulation (transcription is neither suppressed nor induced). In contrast, the term “inducibly expresses” refers to the regulated expression of a gene of interest (transcription occurs in response to an inducer).

As used herein, the term “vector” is used in reference to nucleic acid molecules that transfer DNA segment(s) from one cell to another. Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome-binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression that is higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots).

The terms “transfection” and “transformation” as used herein refer to the introduction of foreign DNA into eukaryotic cells by any method. Transfection may be accomplished by a variety of means known to the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

The term “stable transfection” or “stably transfected” refers to the introduction and integration of foreign DNA into the genome of the transfected cell. The tern “stable transfectant” refers to a cell that has stably integrated foreign DNA into the genomic DNA. The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The term “calcium phosphate precipitation” refers to a technique for the introduction of nucleic acids into a cell. The uptake of nucleic acids by cells is enhanced when the nucleic acid is presented as a calcium phosphate-nucleic acid co-precipitate. The original technique (Graham and van der Eb, Virol, 52:456, 1973), is modified to optimize conditions for particular types of cells.

As used herein, the terms “drug selection marker” and “selectable marker” refer to the use of a gene that encodes an enzymatic activity that confers the ability to grow in medium lacking what would otherwise be an essential nutrient. In addition, a selectable marker may confer resistance to an antibiotic or drug upon the cell in which the selectable marker is expressed. Selectable markers may be “dominant”; a dominant selectable marker encodes an enzymatic activity that can be detected in any eukaryotic cell line (e.g., the bacterial aminoglycoside 3′ phosphotransferase gene or neo gene conferring resistance to the drug G418 in mammalian cells, the bacterial hygromycin G phosphotransferase gene or hyg gene conferring resistance to the antibiotic hygromycin, and the bacterial xanthine-guanine phosphoribosyl transferase gene or gpt gene conferring the ability to grow in the presence of mycophenolic acid). Other selectable markers are not dominant in that there use must be in conjunction with a cell line that lacks the relevant enzyme activity (e.g., the thymidine kinase gene or tk gene that is used in conjunction with tk-negative cell lines, the CAD gene which is used in conjunction with CAD-deficient cells and the mammalian hypoxanthine-guanine phosphoribosyl transferase gene or hprt gene which is used in conjunction with hprt-negative cell lines). A review of the use of selectable markers in mammalian cell lines is provided in Sambrook, et al., (Molecular Cloning. A Laboratory Manual, 2nd ed., Cold Spring Harbor Laboratory Press, New York, pp.16.9-16.15, 1989).

As used herein, the term “cell culture” refers to any in vitro culture of cells. Included within this term are continuous cell lines (e.g., immortal cells), primary cell cultures, finite cell lines (e.g., non-transformed cells), and any other cell population maintained in vitro.

As used herein the term “tetracycline” refers to a broad spectrum antibiotic produced by Streptomyces aureofasciens, that blocks binding of aminoacyl tRNA to the ribosomes of both gram-positive and gram-negative organisms. The term “tetracycline derivative” refers to an antibiotic derived either directly or indirectly (by modification or partial substitution) from tetracycline, including but not limited to demeclocycline and doxycycline.

As used herein the term “physiologically acceptable diluent” refers to a solution appropriate for administration to an animal. This term includes but is not limited to buffers such as saline, phosphate-buffered saline, etc.

The term “alleviating” as used herein, refers to the act of providing relief from some painful state. In some embodiments, the term “alleviating” refers to the lessening of symptoms of schizophrenia. This lessening comprises both sending schizophrenia into remission, and decreasing the severity of the symptoms of schizophrenia. Severity of schizophrenia is measured by any one of several accepted diagnostic measures including but not limited to DSM-IV and ICD-10 (American Psychiatric Association, DSM-IV: Diagnostic and Statistical Manual of Mental Disorders. 4th ed., Washington DC: The Association, 1994; and World Health Organization, The ICD-10 Classification of Mental and Behavioral Disorders. Clinical Descriptions and Diagnostic Guidelines, Geneva: WHO, 1993). Additionally, in some embodiments of the present invention, the term “alleviating” in reference to schizophrenia refers to changes in frontal lobe metabolism as measured by positron emission tomography or single-photon emission computed tomography.

Similarly, the terms “alleviating” or “reducing” when used in reference to drug addiction, refer to the lessening of symptoms associated with addictive disease. In some embodiments, the term “alleviating” comprises complete cessation of drug use, while in other embodiments, the term “alleviating” comprises reducing the frequency or amount of drug used. In preferred embodiments, the term “alleviating” comprises significantly reducing the frequency or amount of drug used as determined by accepted statistical methods.

The term “forebrain” as used herein refers to the anterior of the three principal divisions of the brain.

As used herein, the term “treatment refractory schizophrenia” refers to the presence of ongoing psychotic symptoms with substantial functional disability and/or behavioral deviances that persist in persons diagnosed with schizophrenia, despite reasonable and customary pharmacological and psychosocial treatment that has been provided for an adequate time period (e.g., about two years). Specifically, some embodiments of the present invention utilize Brenner and colleagues definition of treatment-refractory schizophrenia (Brenner et al., Schizophrenia Bull, 16:551-562, 1990).

The term “reinforcing effects” of an addictive drug as used herein includes both positive and negative effects. For instance, in some embodiments, the term “positive reinforcing effect” includes but is not limited to a sense of alcohol making a person feel whole, increasing confidence, increasing sociability and making everything seem right with the world, while the term “negative reinforcing effect” includes but is not limited to reducing anxiety, and/or blocking out unpleasant memories.

### DESCRIPTION OF THE INVENTION

Overactivity of brain dopamine systems in limbic systems of brain is postulated to be a major molecular determinant of schizophrenia. To treat this defect, neural stem cells overexpressing the human dopamine transporter (hDAT) are engineered for transplantation into the forebrains of schizophrenic patients. This strategy is contemplated to reduce extracellular dopamine levels, and thus alter the pathological behavioral manifestations of schizophrenia.

Neural stem cells are pluripotent cells that exist in the developing and adult brain. Neural stem cells have a capacity to differentiate into all known neural cell types including neurons, astrocytes and oligodendrocytes. Unprecedented plasticity of neural stem cells makes them ideal candidates for genetic modification and transplantation into the central nervous system (CNS). As a proof of concept, C17.2 cells, a clonal multipotent neural stem cell line originally derived from the external germinal layer of neonatal mouse cerebellum were employed. C17.2 cells have been immortalized with the v-,nyc oncogene, and stably express β-galactosidase. The growth of C17.2 cells is contact inhibited, and they do not form tumors in nude mice and do not grow in soft agar. C17.2 cells have been previously shown to spontaneously differentiate into cholinergic neurons and astrocytes when transplanted into the septum/diagonal band nuclei of young adult rats and mice.

The synaptic actions of dopamine are attenuated primarily by the plasma membrane dopamine transporter (DAT). The DAT is localized exclusively on dopamine neurons, making it a specific marker for dopamine neurons. The DAT translocates dopamine down its concentration gradient, normally resulting in uptake of dopamine back into dopamine neurons. Thus, the DAT is the primary physiological mechanism for terminating dopamine neurotransmission in the CNS.

Experimental

The following examples are provided in order to demonstrate and further illustrate certain preferred embodiments and aspects of the present invention and are not to be construed as limiting the scope thereof.

In the experimental disclosure which follows, the following abbreviations apply: DA (dopamine); DAT (dopamine transporter); hDAT (human DAT); DOX (doxycycline); EGFP (enhanced green fluorescence protein); eq (equivalents); M (Molar); μM (micromolar); N (Normal); mol (moles); mmol (millimoles); tmol (micromoles); nmol (nanomoles); g (grams); mg (milligrams); μg (micrograms); ng (nanograms); I or L (liters); ml (milliliters); μl (microliters); cm (centimeters); mm (millimeters); pm (micrometers); nm (nanometers); ° C. (degrees Centigrade); U (units), mU (milliunits); min. (minutes); sec. (seconds); % (percent); kb (kilobase); bp (base pair); cpm (counts per minute); Ci (Curies); PCR (polymerase chain reaction); DSM-IV (Diagnostic and Statistical Manual of Mental Disorders—Fourth Edition); and ICD-10 (International Statistical Classification of Diseases and Related Health Problems).

### EXAMPLE 1

### Plasmid Construction

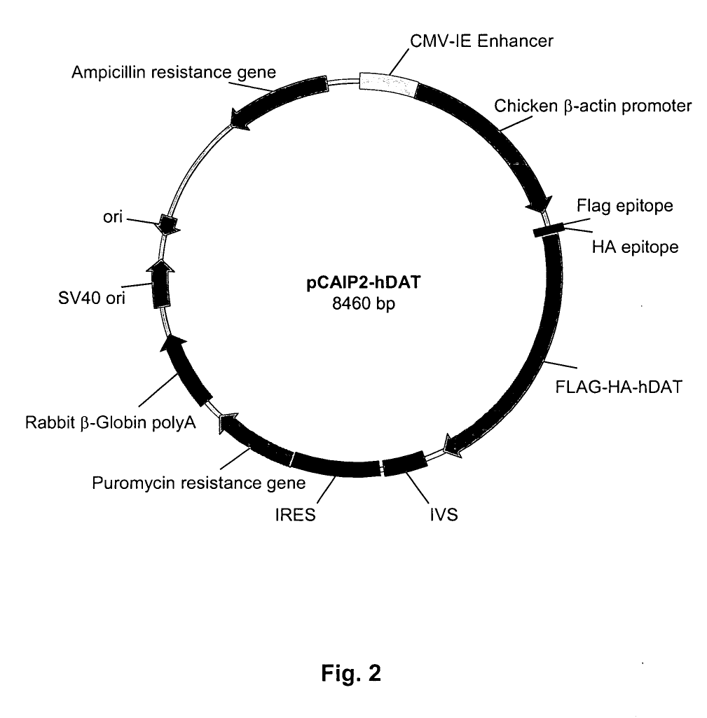

To ensure a strong expression of hDAT in C17.2 cells after transplantation and differentiation, a combination of the cytomegalovirus enhancer and the chicken beta-actin promoter was employed. The murine C17.2 neural stem cells, were kindly provided by E. Snyder (Snyder et al., Cell, 68:33-51, 1992; and Ryder et al., J Neurobiol, 21:356-375, 1990). The mRNA driven from this promoter contains the coding sequence of hDAT, an intron, an internal ribosome entry site, and the coding sequence of the puromycin resistance gene. The hDAT cassette utilized herein contains both HA and FLAG epitopes tags, which were kindly supplied by J. A. Javitch (Saunders et al., Proc Natl Acad Sci USA, 97:6850-6855, 2000). This arrangement shown in the plasmid map for pCAIP2-hDAT of FIG. 2, facilitated screening of stable transfectants expressing hDAT.

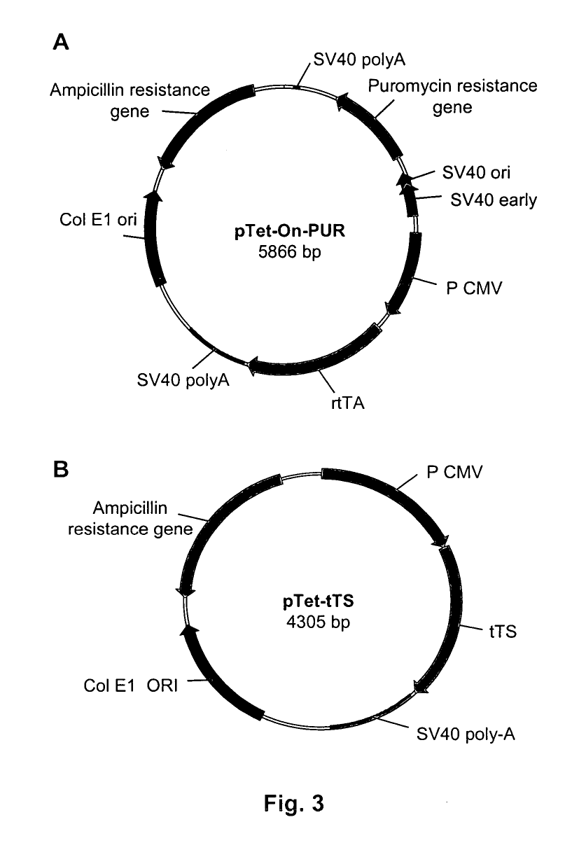

For tightly controlled inducible expression of HDAT, the Tet-On expression system (See, FIG. 3, panel A) was utilized in combination with the tetracycline-controlled transcriptional silencer (tTS; See, FIG. 3, panel B). A major drawback of the Tet-On expression system is unregulated basal transcription. However, the tTS represses transcription from the Tet-On system in the absence of tetracycline, yielding a superior inducible gene expression system.

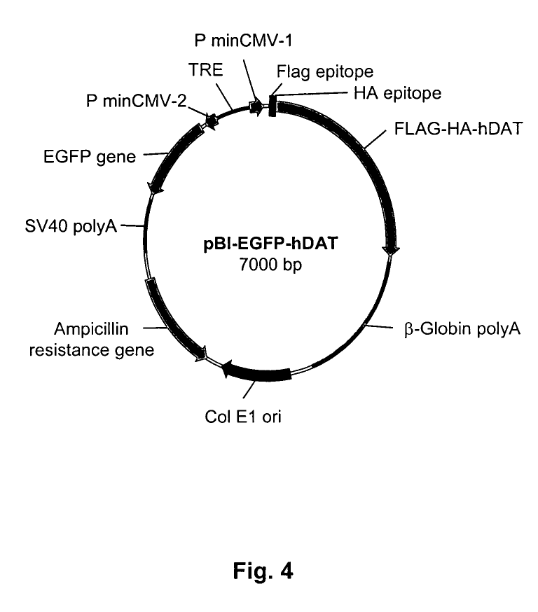

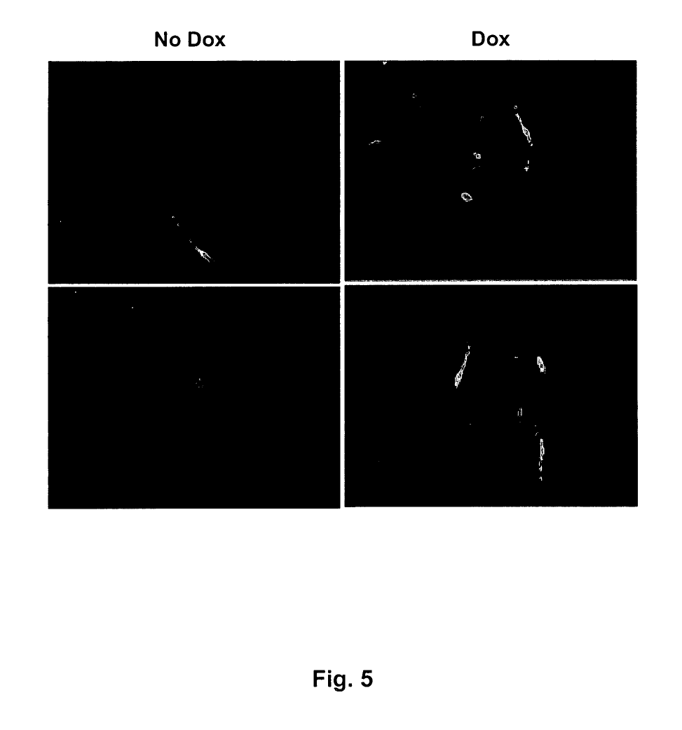

Additionally, an enhanced green fluorescent protein (EGFP) was co-expressed with HDAT, from a bidirectional tetracycline responsive promoter (See, FIG. 4). The EGFP expression was employed to efficiently select transfected cells exhibiting a tight regulation of hDAT expression, through the measurement of fluorescence (See, FIG. 5).

### EXAMPLE 2

### Engineering Neural Stem Cells to Express the Human Dopamine Transporter

This example provides methods for obtaining neural stem cells (NSC) that express the human dopamine transporter (HDAT) in either a constitutive or an inducible fashion. All transfections were done using the calcium phosphate precipitation method. This well known method yielded an approximately 40% transfection efficiency in C17.2 cells. However, the present invention is not limited to this method of transfection, in fact other common methods are suitable for transfecting nucleic acids into stem cells.

Constitutive hDAT-NCS. Murine C17.2 neural stem cells were transfected with the pCAIP2-hDAT expression vector using methods known in the art. Selection of stable transfectants expressing hDAT was done in the presence of puromycin. Approximately 30 puromycin-resistant colonies were then screen for HDAT expression by virtue of the HA and FLAG epitope tags inserted at the amino-terminus of the hDAT molecule. However, the present invention is not limited to HA or FLAG tags, as other tags are suitable for this purpose. Additionally, although epitope tags permit efficient selection of transfected cells, heterologous epitope tags are not required.

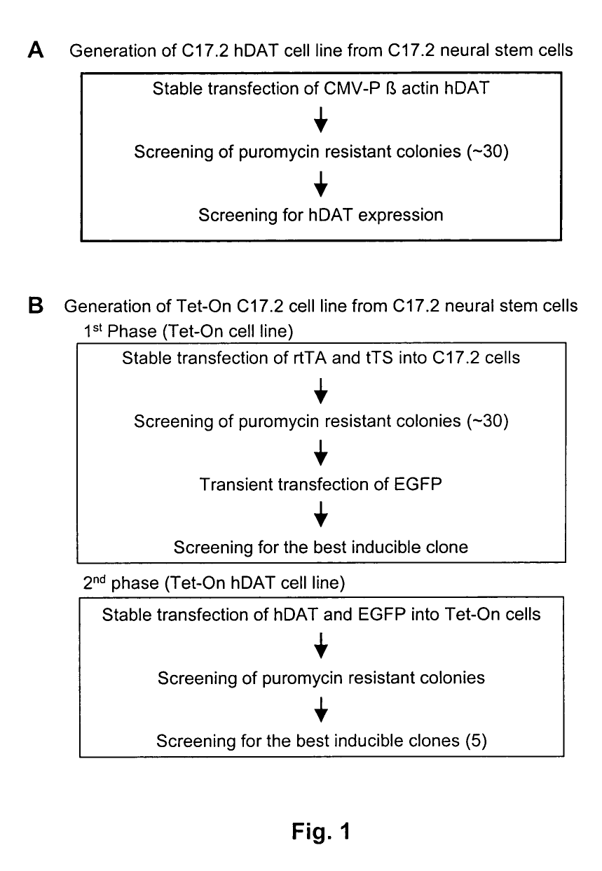

Inducible hDAT-NCS. As shown in FIG. 1, panel B, the generation of Tet-On hDAT cells was done in a two phase process. In the first phase, pTet-On-Pur and pTet-tTS were co-transfected into C17.2 cells. Cells expressing rtTA were selected by culturing the cells in the presence of puromycin. Next, rtTA-expressing cells were transiently transfected with an EGFP expression vector. The best inducible clones were selected on the basis of EGFP expression in the presence of doxycycline. The inducible Tet-On neural stem cells were expanded and co-transfected with the bi-directional EGFP/hDAT expression plasmid, pBI-EGFP-hDAT, containing a puromycin-resistance marker. Stable transfectants were selected in the presence of puromycin, yielding five clones with the desired phenotype (inducible hDAT expression).

### EXAMPLE 3

### Dopamine Transporter Expression by Transfected Neural Stem Cells

Using either the constitutive expression system or the inducible expression system, hDAT expression in C17.2 cells was examined using a tritiated-dopamine (³[H]DA) uptake assay. Transfected neural stem cells were plated in 12-well plates coated with 0.01 mg/ml poly-L-lysine. The cells were preincubated for 30 min in 1 ml of pre-warmed (37° C.), Krebs-Ringer's-HEPES medium (KRH), pH 7.4, containing: 120 mM NaCl, 4.7 mM KCl, 2.2 mM CaCl₂, 1.2 mM MgSO₄, 10 mM HEPES, and 0.18% glucose. Uptake was measured for 10 min at 37° C. by replacing the media with KRH containing 50 nM [³H]DA, 10 μM pargyline and 10 μM pyrocatechol. Uptake was terminated by 3 washes with ice cold KRH, and the cells were lysed with 0.5 ml of 1% SDS per well. Specific [³H]DA uptake was quantified using liquid scintillation counting. Non-specific uptake was determined in the presence of 1 μM GBR 12909, a HDAT blocking agent.

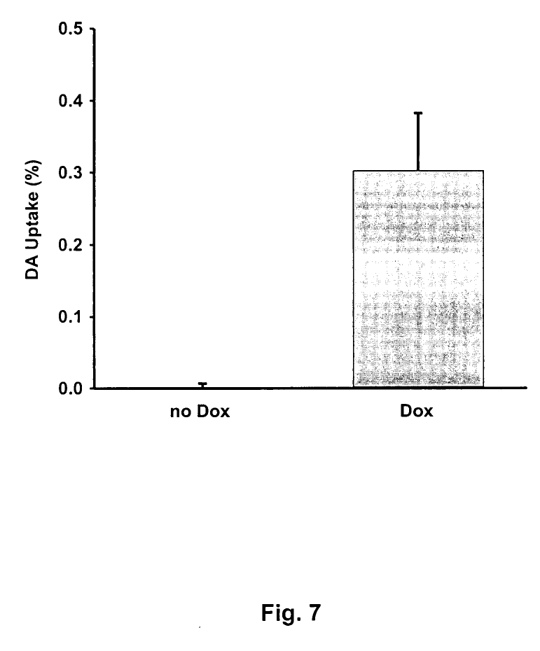

Specifically, the expression of HDAT and EGFP was examined in C17.2 cells transiently transfected with the Tet-On hDAT system. The cells were incubated for 48 hr in the absence (no Dox) or presence (Dox) of 1 μg/ml doxycycline (tetracycline derivative). [³H]DA uptake by non-transfected cells or transfected cells cultured in the absence of doxycycline pre-treatment was not above the background (non-specific uptake determined in the presence of 1 μM GBR 12909). However, in the presence of doxycycline the transfected cells exhibited an ˜30 fold higher [³H]DA uptake over background (See, FIG. 7).

Additionally, hDAT expression was extrapolated from the measurement of EGFP epifluorescence in cells transfected with the EGFP/hDAT bidirectional expression plasmid. As shown in FIG. 5, expression of EGFP was also markedly increased by doxycycline treatment.

### EXAMPLE 4

### Implantation of hDAT-Neural Stem Cells in Rodent Models

The hDAT-neural stem cells obtained using the methods of the preceding examples are tested in one or more strains of mice.

Dopamine Transporter Knock-Out Mice. The hDAT-neural stem cells of the present invention are tested in dopamine transporter knock-out mice (Giros et al., Nature, 379:606-612, 1996). In one embodiment, injections of the hDAT-neural stem cells are made with sterilized 10 μl Hamilton syringes having 23-27 gauge needles. The syringe, loaded with cells, is mounted directly into the head of a stereotaxic frame. The injection needle is lowered to predetermined coordinates through small burr holes in the cranium. Approximately 4.0 μl of the suspension is deposited at the rate of about 1-2 μl/min and a further 2-5 minutes are allowed for diffusion prior to slow retraction of the needle. If desired, multiple deposits can be made along the same needle penetration. The injection may be performed manually or by an infusion pump. At the completion of surgery following retraction of the needle, the mouse is removed from the frame and the wound is sutured. Prophylactic antibiotics or immunosuppressive therapy may be administered as needed.

The success of the transplant is tested by measuring changes in the animal's behavior, changes in the brain uptake of dopamine by various in vivo or in vitro methods, and/or by the analysis of hDAT protein expression by immunohistochemistry or western blotting techniques which are well known in the art.

Inbred Mice With A Natural Preference For Ethanol. As a proof of concept, the hDAT-neural stem cells of the present invention were transplanted into the nucleus accumbens of C57BL/6 mice. Dopamine systems in this area of brain regulate alcohol consumption and other behaviors. Stably-transfected cells with the highest level of dopamine uptake in vitro, assayed as described, were used for transplant, while naive C17.2 cells (not transfected with the hDAT construct) were used as negative controls. Transplant recipients were young adult female mice. Surgeries were performed under isoflurane anesthesia. Approximately 1×10⁶ viable C17.hDAT cells in a volume of 2 μl were injected with a 24 gauge stainless steel cannula attached to a microliter syringe. The syringe was mounted directly into the head of a stereotaxic frame and the injection needle was lowered through small burr holes in the cranium to coordinates based on the mouse brain atlas of Franklin and Paxinos (Franklin and Paxinos, The Mouse Brain in Stereotaxic Coordinates, Academic Press, San Diego, 1997): +1.54 mm AP from bregma; +1.54 mm from midline; and −3.80 to −4.80 mm VD below bregma. Cells were implanted at a rate of 1 μl/minute and the injection needle was pulled out of the brain at a rate of 1 mm/minute using a motorized arm. To equalize pressure around the transplant cannula, withdrawal of the injection needle was halted for two minutes and the cannula was slowly withdrawn from the brain with the pump on. All transplants were bilateral to assure maximal effect and to avoid any unilateral biases or compensations that might be elicited by unilateral transplants. At the completion of surgery, following retraction of the needle, each mouse was removed from the frame and the wound was sutured. Prophylactic antibiotics were administered.

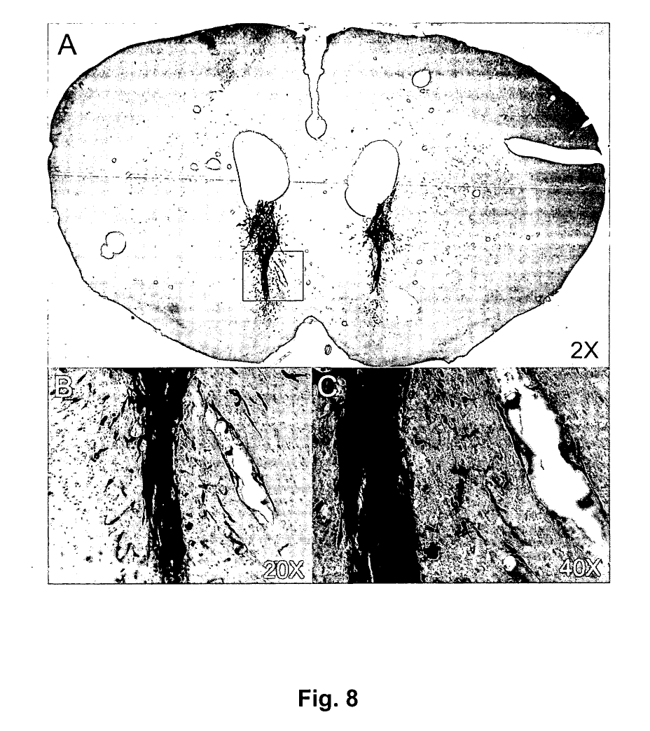

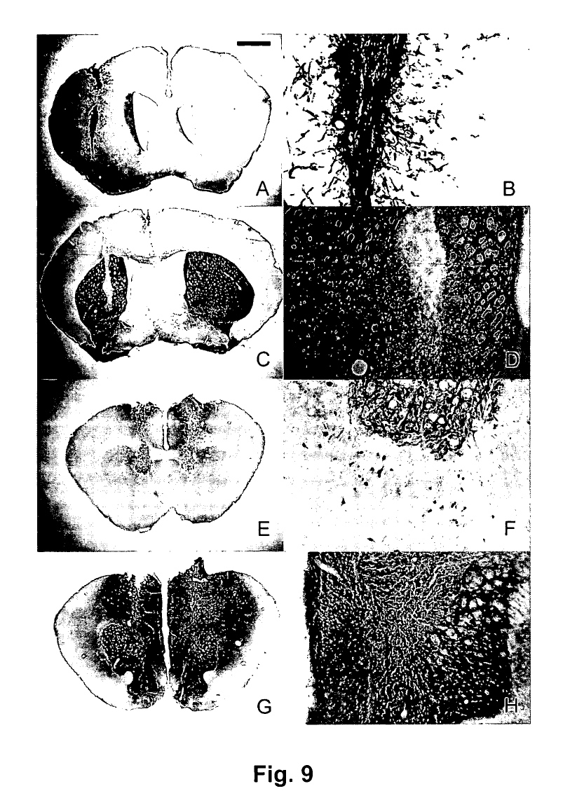

The success of the transplant was assessed by immunohistochemistry seven days following transplantation. Animals were killed by an overdose of anesthetic followed by cardiac perfusion with cold heparinized saline and 4% paraformaldehyde. Brains were removed, placed in 4% paraformaldehyde for two days and then cryopreserved in 30% sucrose/phosphate buffer for at least 24 hours. Brains were sectioned on a cryostat at 40 μm intervals and all sections through the transplant tract were mounted on gelatin-coated glass slides. Endogenous peroxidase was inactivated by treatment with hydrogen peroxide, while nonspecific binding was blocked with goat serum. The number of transplanted cells was determined by visualizing the X-gal positive cells, or by visualizing the beta-galactosidase and hDAT immunoreactive cells in the transplants, as all the C17.2 cells stably express the marker beta-galactosidase. Human DAT and beta-galactosidase immunoreactivities were determined using polyclonal rabbit antibodies against hDAT (1:200, Santa Cruz) or beta-galactosidase (3′-5′), and biotinylated anti-rabbit IgG secondary antibodies. Sections were incubated with avidin/biotinylated horseradish peroxidase complex (ABC staining kit, Vector) and peroxidase activity was visualized with diaminobenzidine (Pierce). Beta-galactosidase activity was assessed by treating sections with X-gal (5-bromo-4-chloro-3-indoxyl-beta-D-galactopyranoside, 1 mg/ml; Gold Biotechnology). FIG. 8 shows transplanted cells one week after grafting, identified by X-gal staining, in a coronal section of a transplanted brain at the level of the nucleus accumbens. As shown in the higher magnification images, some transplanted cells have migrated slightly outside of the transplant tract. FIG. 9 shows that one week after grafting, C17.2 HDAT cells express hDAT in the grafts, while control (naive) C17.2 cells do not.

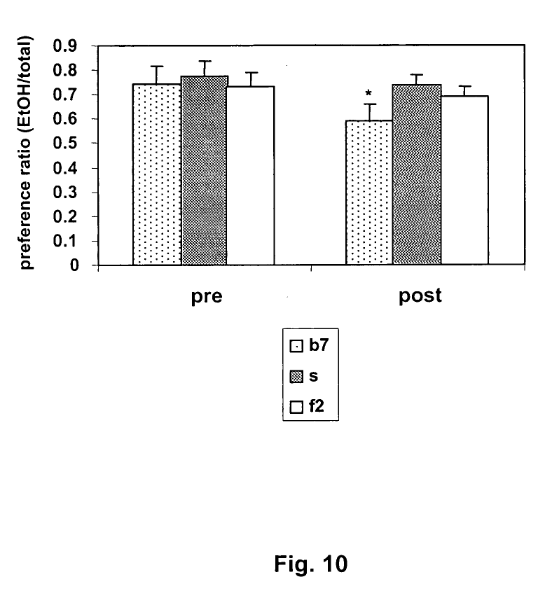

The success of the transplant in changing the behavior of the animals vis-a-vis ethanol, was assessed by measuring changes in ethanol consumption and/or preference. Prior to transplant, ethanol preference was determined for all animals, such that each animal could serve as its own control. Ethanol preference was determined using a two bottle choice paradigm. Individually housed mice were given free access to two water bottles and food ad libitum. After 3 days of exposure to two bottles containing water, one water bottle was replaced with a bottle containing 3% ethanol. Ethanol and water consumption and body weight were measured daily for 3 days and every 3 days thereafter. The position of the bottles was switched at every measurement. The ethanol concentration was raised to 10% after 3 days. Ethanol preference was calculated as the ratio of ethanol solution to total fluid consumed. FIG. 10 shows that when preference for the 10% ethanol solution was determined over a 3-day period (days 3-6) after surgery (“post”), there was a significantly (*p<0.05, paired t-test) lower level of preference in animals that had received the C17.2 HDAT cells (bar labeled b7), in comparison to sham-operated animals (bar labeled s) or animals that received hDAT-negative C17.2 cells (bar labeled f2). In another comparison, when preference for ethanol at times before the transplant surgery (“pre”) was compared to preference after surgery (“post”), the post-transplant ethanol preference was also lower in C17.2 hDAT-transplanted animals compared to the preference of these same animals measured prior to transplantation.

### EXAMPLE 5

### Implantation of hDAT-Neural Stem Cells in Human Patients

The hDAT-neural stem cells obtained using methods of the preceding examples are used to treat patients with severe drug treatment-refractory schizophrenia (as defined by Brenner et al., Schizophrenia Bull, 16:551-562,1990).

Briefly, hDAT-neural stem cells are implanted into the brains of treatment-refractory schizophrenia patients using methods similar to that described for the treatment of Parkinson's patients with human stem cell grafts, as previously described (Freed et al., N Engl J Med, 344:710-719, 2001). The placement of the stem cells is tailored to the treatment of schizophrenia and/or addictive disorders. The patient is given only local anesthesia in order to monitor the patient's ability to speak and move their extremities after each injection. The efficacy of the transplants is measured by analysis of gross clinical improvement or deterioration.

In one embodiment, a stereotactic surgical technique is performed using a CRW computed tomographic (CT) or magnetic resonance (MR) stereotaxic guide (Radionics, Burlington, Mass.). On the day of surgery, the stereotactic head ring is applied to the patient's head under local anesthesia. With the head ring in place, the patient undergoes CT or MR scanning. Baseline coordinates are established for the forebrain. Local anesthesia is used on the skin of the forehead. Incisions 1 cm in length are made in the skin. Implantation is carried out through two 3 mm twist drill holes in the forehead on each side of the midline, one above the other, both below the hairline, and both above the frontal sinus. The patient is awake but sedated with intravenously administered drugs such as midazolam.

All patients are admitted to the recovery room for postoperative observations. Postoperative CT or MR scans are taken to show evidence of hemorrhage. A follow-up appointment for suture removal is made at four to five days after surgery. All patients receive broad-spectrum antibiotics for three days. Phenytoin is administered as prophylaxis against seizures for three days after surgery.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in molecular biology, genetics, medicine, surgery, psychiatry or related fields, are intended to be within the scope of the following claims.

1. A composition comprising a neural stem cell engineered to express a mammalian dopamine transporter.

2. The composition of claim 1, wherein said neural stem cell constitutively expresses said dopamine transporter.

3. The composition of claim 1, wherein said neural stem cell inducibly expresses said dopamine transporter.

4. The composition of claim 1, wherein said dopamine transporter is a human transporter comprising the amino acid sequence set forth as SEQ ID NO:1.

5. The composition of claim 1, further comprising a physiologically acceptable diluent.

6. A method of producing a neural stem cell which inducibly expresses a dopamine transporter, comprising:
a. providing:
i) at least one neural stem cell,
ii) an expression vector comprising a dopamine transporter nucleic acid sequence in operable combination with a tetracycline-responsive transcription activator and a promoter, and
iii) at least one inducible vector comprising a tetracycline-responsive transcription activator and a tetracycline-controlled transcriptional silencer; and
b. contacting said at least one neural stem cell with said expression vector and said at least one inducible vector under conditions suitable for producing a dopamine transporter positive neural stem cell which inducibly expresses said dopamine transporter.

7. The method of claim 6, wherein said dopamine transporter is a human dopamine transporter comprising the amino acid sequence set forth as SEQ ID NO:1.

8. The method of claim 6, wherein at least one of said vectors further comprises a drug selection marker.

9. The method of claim 6, wherein said dopamine transporter-positive neural stem cell internalizes dopamine after growth in the presence of an inducer selected from the group consisting of tetracycline and a tetracycline derivative.

10. The method of claim 6, wherein said dopamine transporter-positive neural stem cell is suitable graft material for human transplantation.

11. A method of alleviating at least one symptom of schizophrenia in a schizophrenic patient, comprising:
a. providing a neural stem cell engineered to inducibly express a human dopamine transporter; and
b. transplanting said neural stem cell into the forebrain of a schizophrenic patient, under conditions suitable for alleviating at least one symptom of schizophrenia.

12. The method of claim 11, wherein said at least one symptom comprises a symptom selected from the group consisting of a positive symptom, a negative symptom, a cognitive symptom, and a mood symptom.

13. The method of claim 12, wherein said positive symptom is selected from the group consisting of delusions, hallucinations, disorganized speech, and catatonia.

14. The method of claim 12, wherein said negative symptom is selected from the group consisting of affective flattening, alogia, avolition and anhedonia.

15. The method of claim 12, wherein said cognitive symptom is selected from the group consisting of an attention deficit, poor memory recall, and a reduced executive function.

16. The method of claim 12, wherein said mood symptom is selected from the group consisting of dysphoria, suicidality, and hopelessness.

17. The method of claim 11, wherein said patient has drug treatment-refractory schizophrenia.

18. The method of claim 11, wherein said alleviating is assessed by DSM-IV diagnostic criteria or ICD-10 diagnostic criteria.

19. A method of reducing the reinforcing effects of an addictive drug in a patient with an addictive disease comprising:
a. providing a neural stem cell engineered to inducibly express a human dopamine transporter; and
b. transplanting said neural stem cell into the brain of a patient with an addictive disease, under conditions suitable for reducing the reinforcing effects of an addictive drug.

20 The method of claim 19, wherein said addictive drug is selected from the group consisting of cocaine, an opiate, nicotine, a cannabinoid, a barbiturate, a benzodiazepine, and alcohol.

21 The method of claim 19, wherein said addictive disease is life-disrupting or life-threatening.

22. The method of claim 19, wherein said reducing comprises lessening the amount of said addictive drug intake by said patient.

23. The method of claim 19, wherein said reducing comprises lessening the frequency of said addictive drug intake by said patient.


SEQUENCE LISTING (2 sequences)

SEQ ID NO: 1 - protein, 620 aa, Homo sapiens
MSKSKCSVGLMSSVVAPAKEPNAVGPKEVELILVKEQNGVQLTSSTLTNPRQSPVEAQDRETWGKKIDFLLSVIGFAVDLANVWRFPYLCYKNGGGAFLVPYLLFMVIAGMPLFYMELALGQFNREGAAGVWKICPILKGVGFTVILISLYVGFFYNVIIAWALHYLFSSFTTELPWIHCNNSWNSPNCSDAHPGDSSGDSSGLNDTFGTTPAAEYFERGVLHLHQSHGIDDLGPPRWQLTACLVLVIVLLYFSLWKGVKTSGKVVWITATMPYVVLTALLLRGVTLPGAIDGIRAYLSVDFYRLCEASVWIDAATQVCFSLGVGFGVLIAFSSYNKFTNNCYRDAIVTTSINSLTSFSSGFVVFSFLGYMAQKHSVPIGDVAKDGPGLIFIIYPEAIATLPLSSAWAVVFFIMLLTLGIDSAMGGMESVITGLIDEFQLLHRHRELFTLFIVLATFLLSLFCVTNGGIYVFTLLDHFAAGTSILFGVLIEAIGVAWFYGVGQFSDDIQQMTGQRPSLYWRLCWKLVSPCFLLFVVVVSIVTFRPPHYGAYIFPDWANALGWVIATSSMAMVPIYAAYKFCSLPGSFREKLAYAIAPEKDRELVDRGEVRQFTLRHWLKV

SEQ ID NO: 2 - DNA, 3946 bp, Homo sapiens
accgctccggagcgggaggggaggcttcgcggaacgctctcggcgccaggactcgcgtgcaaagcccaggcccgggcggccagaccaagagggaagaagcacagaattcctcaactcccagtgtgcccatgagtaagagcaaatgctccgtgggactcatgtcttccgtggtggccccggctaaggagcccaatgccgtgggcccgaaggaggtggagctcatccttgtcaaggagcagaacggagtgcagctcaccagctccaccctcaccaacccgcggcagagccccgtggaggcccaggatcgggagacctggggcaagaagatcgactttctcctgtccgtcattggctttgctgtggacctggccaacgtctggcggttcccctacctgtgctacaaaaatggtggcggtgccttcctggtcccctacctgctcttcatggtcattgctgggatgccacttttctacatggagctggccctcggccagttcaacagggaaggggccgctggtgtctggaagatctgccccatactgaaaggtgtgggcttcacggtcatcctcatctcactgtatgtcggcttcttctacaacgtcatcatcgcctgggcgctgcactatctcttctcctccttcaccacggagctcccctggatccactgcaacaactcctggaacagccccaactgctcggatgcccatcctggtgactccagtggagacagctcgggcctcaacgacacttttgggaccacacctgctgccgagtactttgaacgtggcgtgctgcacctccaccagagccatggcatcgacgacctggggcctccgcggtggcagctcacagcctgcctggtgctggtcatcgtgctgctctacttcagcctctggaagggcgtgaagacctcagggaaggtggtatggatcacagccaccatgccatacgtggtcctcactgccctgctcctgcgtggggtcaccctccctggagccatagacggcatcagagcatacctgagcgttgacttctaccggctctgcgaggcgtctgtttggattgacgcggccacccaggtgtgcttctccctgggcgtggggttcggggtgctgatcgccttctccagctacaacaagttcaccaacaactgctacagggacgcgattgtcaccacctccatcaactccctgacgagcttctcctccggcttcgtcgtcttctccttcctggggtacatggcacagaagcacagtgtgcccatcggggacgtggccaaggacgggccagggctgatcttcatcatctacccggaagccatcgccacgctccctctgtcctcagcctgggccgtggtcttcttcatcatgctgctcaccctgggtatcgacagcgccatgggtggtatggagtcagtgatcaccgggctcatcgatgagttccagctgctgcacagacaccgtgagctcttcacgctcttcatcgtcctggcgaccttcctcctgtccctgttctgcgtcaccaacggtggcatctacgtcttcacgctcctggaccattttgcagccggcacgtccatcctctttggagtgctcatcgaagccatcggagtggcctggttctatggtgttgggcagttcagcgacgacatccagcagatgaccgggcagcggcccagcctgtactggcggctgtgctggaagctggtcagcccctgctttctcctgttcgtggtcgtggtcagcattgtgaccttcagacccccccactacggagcctacatcttccccgactgggccaacgcgctgggctgggtcatcgccacatcctccatggccatggtgcccatctatgcggcctacaagttctgcagcctgcctgggtcctttcgagagaaactggcctacgccattgcacccgagaaggaccgtgagctggtggacagaggggaggtgcgccagttcacgctccgccactggctcaaggtgtagagggagcagagacgaagaccccaggaagtcatcctgcaatgggagagacacgaacaaaccaaggaaatctaagtttcgagagaaaggagggcaacttctactcttcaacctctactgaaaacacaaacaacaaagcagaagactcctctcttctgactgtttacacctttccgtgccgggagcgcacctcgccgtgtcttgtgttgctgtaataacgacgtagatctgtgcagcgaggtccaccccgttgttgtccctgcagggcagaaaaacgtctaacttcatgctgtctgtgtgaggctccctccctccctgctccctgctcccggctctgaggctgccccaggggcactgtgttctcaggcggggatcacgatccttgtagacgcacctgctgagaatccccgtgctcacagtagcttcctagaccatttactttgcccatattaaaaagccaagtgtcctgcttggtttagctgtgcagaaggtgaaatggaggaaaccacaaattcatgcaaagtcctttcccgatgcgtggctcccagcagaggccgtaaattgagcgttcagttgacacattgcacacacagtctgttcagaggcattggaggatgggggtcctggtatgtctcaccaggaaattctgtttatgttcttgcagcagagagaaataaaactccttgaaaccagctcaggctactgccactcaggcagcctgtgggtccttgtggtgtagggaacggcctgagaggagcgtgtcctatccccggacgcatgcagggcccccacaggagcgtgtcctatccccggacgcatgcagggcccccacaggagcatgtcctatccctggacgcatgcagggcccccacaggagcgtgtactaccccagaacgcatgcagggcccccacaggagcgtgtactaccccaggacgcatgcagggcccccactggagcgtgtactaccccaggacgcatgcagggcccccacaggagcgtgtcctatccccggaccggacgcatgcagggcccccacaggagcgtgtactaccccaggacgcatgcagggcccccacaggagcgtgtactaccccaggatgcatgcagggcccccacaggagcgtgtactaccccaggacgcatgcagggcccccatgcaggcagcctgcagaccaacactctgcctggccttgagccgtgacctccaggaagggaccccactggaattttatttctctcaggtgcgtgccacatcaataacaacagtttttatgtttgcgaatggctttttaaaatcatatttacctgtgaatcaaaacaaattcaagaatgcagtatccgcgagcctgcttgctgatattgcagtttttgtttacaagaataattagcaatactgagtgaaggatgttggccaaaagctgctttccatggcacactgccctctgccactgacaggaaagtggatgccatagtttgaattcatgcctcaagtcggtgggcctgcctacgtgctgcccgagggcaggggccgtgcagggccagtcatggctgtcccctgcaagtggacgtgggctccagggactggagtgtaatgctcggtgggagccgtcagcctgtgaactgccaggcagctgcagttagcacagaggatggcttccccattgccttctggggagggacacagaggacggcttccccatcgccttctggccgctgcagtcagcacagagagcggcttccccattgccttctggggagggacacagaggacagtttccccatcgccttctggttgttgaagacagcacagagagcggcttccccatcgccttctggggaggggctccgtgtagcaacccaggtgttgtccgtgtctgttgaccaatctctattcagcatcgtgtgggtccctaagcacaataaaagacatccacaatggaaaaaaaaaaaggaattc